(19) United States
(12) Patent Application Publication
(10) Pub. No.: US 2005/0222467 A1
(43) Pub. Date: Oct. 6, 2005

(54) Process for production of bisphenol a

(76) Inventors: Masahiro Kodama, Chiba (JP); Kazuyuki Hirano, Chiba (JP); Norio Ogata, Chiba (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/508,012

(22) PCT Filed: Mar. 19, 2003
(86) PCT No.: PCT/JP03/03330

§ 371 Date: Apr. 19, 2005

(30) Foreign Application Priority Data
Mar. 29, 2002 (JP) 2002-96701

(51) Int. Cl. C07C 39/16

(52) U.S. Cl. 568/728

(57) ABSTRACT

In the process for producing bisphenol A comprising crystallizing an adduct of bisphenol A and phenol from a phenol solution of bisphenol A obtained by reacting phenol and acetone in the presence of an acid catalyst, treating the formed slurry by solid-liquid separation and removing phenol from the solid component, a layer of the crystalline adduct is formed on a filter by filtering slurry (1) comprising the crystalline adduct, washing the layer of the adduct with a washing liquid, dissolving the layer of the adduct in a solution comprising phenol, producing slurry (2) comprising the crystalline adduct by crystallization, and separating the crystalline adduct by centrifugation of slurry (2). When bisphenol A is separated from the reaction mixture, the adduct of bisphenol A and phenol can be recovered from the mother liquor of the reaction efficiently at a high purity in accordance with the above process.

### TECHNICAL FIELD

The present invention relates to a process for producing bisphenol A [2,2-bis(4-hydroxyphenyl)propane] and, more particularly, to a process for separating an adduct of bisphenol A and phenol in said process.

### BACKGROUND ART

It is known that bisphenol A is an important compound as the material for engineering plastics such as polycarbonate resins and polyarylate resins and epoxy resins and the demand for bisphenol A is increasing.

Bisphenol A is produced by condensation of phenol in an excess amount and acetone in the presence of an acidic catalyst and a cocatalyst such as a sulfur compound which is used occasionally.

As the process for separating bisphenol A from the reaction mixture, a process of separating bisphenol A directly from the reaction mixture in a crude crystalline condition and a process of separating an adduct of bisphenol A and phenol as crystals by concentrating and cooling a liquid mixture which is obtained by removing acetone and water from the reaction mixture (Japanese Patent Application Laid-Open Nos. Showa 51(1976)-91240 and 57(1982)-77637), have been known.

The process of separating an adduct directly from the reaction mixture in a crude crystalline condition has a drawback in that washing must be conducted repeatedly due to the finely crystalline condition of bisphenol A and the loss increases.

Therefore, the process of separating the adduct of bisphenol A and phenol as crystals is mainly used. In this process, the adduct of bisphenol A and phenol is crystallized and the formed crystals are separated from the mother liquor in accordance with a conventional process of solid-liquid separation using a filter or a centrifuge (Japanese Patent Application Laid-Open Nos. Showa 57(1982)-77637, Heisei 5(1993)-331088, Showa 63(1988)-275539, Heisei 6(1994)-107578 and Heisei 6(1994)-306002).

In the above process of solid-liquid separation using the filtration, a belt filter of the suction type or a drum filter can be used (Japanese Patent Application Laid-Open No. Heisei 6(1994)-306002). However, it is necessary that the size of the crystals be increased and the surface area be decreased so that the loss through the opening of the filter material is prevented and the purity is enhanced (Japanese Patent Application Laid-Open No. Heisei 5(1993)-331088). The filtration has drawbacks in that the content of the liquid is great and it is difficult that the mother liquor contained between the crystals is removed sufficiently, that there is the possibility that the mother liquor is contained within crystals having great sizes, and that, when the size of the crystals is decreased to overcome the above drawback, the efficiency of the filtration decreases markedly due to difficulty in passage of the mother liquor through the layer of the crystals and clogging of the filter.

The process using the centrifuge (Japanese Patent Application Laid-Open Nos. Heisei 6(1994)-107578 and Heisei 6(1994)-306002) has the advantage in that the content of the liquid between the crystals is decreased and an adduct containing less liquids can be obtained. However, fragmentation of the crystals takes place due to the load of centrifugation and the efficiency of substitution of the mother liquor and the washing liquid is inferior to that of the filtration. Therefore, in the treatment of a great amount of the product, in general, it is essential for improving the purity that the washing is conducted repeatedly by using a plurality of apparatuses. The number of the apparatuses increases and the time of operation also increases. Therefore, this process is not preferable from the standpoint of economy.

The crystallization in a plurality of steps to improve the purity is described in Japanese Patent Application Laid-Open Nos. Heisei 1(1989)-230538 and Heisei 7(1995)-257978).

### DISCLOSURE OF THE INVENTION

The present invention has an object of providing a process for producing bisphenol A which enables recovering the adduct of bisphenol A and phenol efficiently at a high purity when bisphenol A is separated from the reaction mixture.

As the result of intensive studies by the present inventors to overcome the above difficulties, it was found that a high purity adduct of bisphenol A and phenol could be obtained when the crystallization was conducted in the following two steps. In the first step, filtration was used for the separation so that impurities on the surface of the crystals were removed and, in the second step, the crystals were dissolved, recrystallized and centrifuged and the washing liquid was sufficiently removed so that impurities at the inside of the crystals were removed. The obtained product was used in the steps which followed. The present invention has been completed based on this knowledge.

The present invention provides a process for producing bisphenol A which comprises crystallizing an adduct of bisphenol A and phenol from a phenol solution of bisphenol A which is obtained by reacting phenol and acetone in a presence of an acid catalyst, treating a formed slurry by solid-liquid separation and removing phenol from a solid component, wherein the process comprises forming a layer of the adduct of bisphenol A and phenol in a crystalline condition on a filter by filtering phenol slurry of bisphenol A (1) comprising the adduct of bisphenol A and phenol in a crystalline condition with the filter, washing the layer of the adduct with a washing liquid, dissolving the layer of the adduct obtained after the washing in a solution comprising phenol, producing phenol slurry of bisphenol A (2) comprising the adduct of bisphenol A and phenol in a crystalline condition by crystallization, and separating the adduct of bisphenol A and phenol in a crystalline condition by centrifugation of phenol slurry of bisphenol (2).

### THE MOST PREFERRED EMBODIMENT TO CARRY OUT THE INVENTION

In the process for producing bisphenol A of the present invention, bisphenol A is produced in accordance with (A) a step of reaction of phenol and acetone, (B) a step of removal of water formed by the reaction and substances having low boiling points of the unreacted raw materials, (C) a step of concentration of bisphenol A, (D) a step of crystallization and solid-liquid separation, (E) a step of dissolution, crystallization and solid-liquid separation of the adduct of bisphenol A and phenol, (F) a step of melting by heating, (G) a step of removal of phenol from bisphenol A, and (H) a step of granulation.

Each step in the process for producing bisphenol A will be described in the following.

(A) Step of Reaction

In the step of reaction, phenol in an excess amount and acetone are condensed in the presence of an acidic catalyst and bisphenol A is formed. As the acidic catalyst, an acid-type ion exchange resin can be used. The acid-type ion exchange resin is not particularly limited and acid-type ion exchange resins conventionally used as the catalyst for producing bisphenol A can be used. From the standpoint of the catalyst activity, cation exchange resins of the sulfonic acid type are preferable.

The cation exchange resin of the sulfonic acid type is not particularly limited as long as the cation exchange resin of the sulfonic acid type is a strongly acidic cation exchange resin having sulfonic acid group. Examples of the cation exchange resin of the sulfonic acid type include sulfonated copolymers of styrene and divinylbenzene, sulfonated crosslinked styrene polymers, phenol-formaldehyde-sulfonic acid resins and benzene-formaldehyde-sulfonic acid resins. The above resin may be used singly or in combination of two or more.

In the present step, in general, a mercaptan is used as the cocatalyst in combination with the acid-type ion exchange resin. The mercaptan is a compound having SH group in the free form in the molecule. As the mercaptan, alkylmercaptans and alkyl mercaptans having at least one substituent such as carboxyl group, amino group and hydroxyl group such as mercaptocarboxylic acids, aminoalkanethiol and mercaptoalcohols, can be used. Examples of the mercaptan include alkylmercaptans such as methylmercaptan, ethylmercaptan, n-butylmercaptan and n-octylmercaptan; thiocarboxylic acids such as thioglycolic acid and β-mercaptopropionic acid; aminoalkanethiols such as 2-aminoethanethiol; and mercaptoalcohols such as mercaptoethanol. Among these mercaptans, alkylmercaptans are preferable from the standpoint of the effect as the cocatalyst. The mercaptan may be used singly or in combination of two or more.

The mercaptan may be fixed to the acid-type ion exchange resin and used as the cocatalyst.

The amount of the mercaptan is selected, in general, in the range of 0.1 to 20% by mole and preferably in the range of 1 to 10% by mole based on the amount of acetone used as the raw material.

The relative amounts of phenol and acetone are not particularly limited. From the standpoint of the easiness of purification of the formed bisphenol A and economy, it is preferable that the amount of unreacted acetone is as small as possible. Therefore, it is advantageous that phenol is used in an amount greater than the stoichiometric amount. In general, 3 to 30 moles and preferably 5 to 15 moles of phenol is used per 1 mole of acetone. In the production of bisphenol A, in general, no solvent is necessary for the reaction unless the viscosity of the reaction liquid is excessively great or the reaction is conducted at a low temperature such that the operation of the reaction becomes difficult due to freezing.

The condensation reaction of phenol and acetone may be conducted in accordance with any of the batch process and the continuous process. It is advantageous that the fixed bed continuous process in which the reaction is conducted while phenol, acetone and a mercaptan (when the mercaptan is not fixed to an acid-type ion exchange resin) are continuously supplied to a reaction column packed with the acid-type ion exchange resin, is used. In the reaction, a single reaction column or a plurality of the reaction columns connected in series may be used. Industrially, it is advantageous that the fixed bed multi-stage continuous process using at least two reaction columns which are packed with an acid-type ion exchange resin and connected in series, is conducted.

The reactions condition in the fixed bed continuous process will be described in the following.

The ratio of the amount by mole of acetone to the amount by mole of phenol is, in general, selected in the range of 1/30 to 1/3 and preferably in the range of 1/15 to 1/5. When the ratio of the amounts is smaller than 1/30, there is the possibility that the reaction is excessively slow. When the ratio of the amounts exceeds 1/3, there is the tendency that the amount of formed impurities increases and the selectivity of bisphenol A decreases. When the mercaptan is not fixed to the acid-type ion exchange resin, the ratio of the amount by mole of mercaptan to the amount by mole of acetone is selected, in general, in the range of 0.1/100 to 20/100 and preferably in the range of 1/100 to 10/100. When the ratio of the amounts is smaller than 0.1/100, there is the possibility that the effect of improving the reaction rate and the selectivity of bisphenol A is not sufficiently exhibited. When the ratio of the amounts exceeds 20/100, the effect is not exhibited to the degree expected from the amount.

The reaction temperature is selected, in general, in the range of 40 to 150° C. and preferably in the range of 60 to 110° C. When the reaction temperature is lower than 40° C., the reaction is slow. Moreover, the viscosity of the reaction fluid is great and there is the possibility that the reaction system is solidified, occasionally. When the reaction temperature exceeds 150° C., controlling the reaction becomes difficult. Moreover, the selectivity of bisphenol A (p,p′-compound) decreases and the acid-type ion exchange resin of the catalyst may be decomposed or degraded, occasionally. LHSV (the liquid hourly space velocity) of the mixture of the raw materials is selected, in general, in the range of 0.2 to 30 hr⁻¹ and preferably in the range of 0.5 to 10 hr⁻¹.

(B) Step of Removal of Substances having Low Boiling Points

In the step of removal of substances having low boiling points, the reaction mixture containing bisphenol A which is obtained in the step of reaction of step (A) is treated for removal of substances having low boiling points. In the batch process, the step is conducted in the substantial absence of the acid-type ion exchange resin, i.e., after removal of the acid-type ion exchange resin by filtration or the like. In the fixed bed continuous process, the step is conducted without any further treatments.

In the present step, in general, substances having low boiling points such as unreacted acetone, water formed as the byproduct and alkylmercaptane are removed by distillation under a reduced pressure using a distillation column.

The distillation under a reduced pressure is conducted, in general, in the condition of a pressure of 6.5 to 80 kPa and a temperature of 70 to 180° C. In the distillation, unreacted phenol forms an azeotrope and a portion of the unreacted phenol is removed to the outside of the system from the top of the column in combination with the substances having low boiling points. In this distillation, it is desirable that the temperature of the source of heating is kept at 190° C. or lower so that heat decomposition of bisphenol A is prevented. As the material of the apparatus, in general, SUS304, SUS316 and SUS316L are used.

(C) Step of Concentration

The liquid at the bottom of the column obtained by removing substances having low boiling points from the reaction mixture contains bisphenol A and phenol. By distillation under a reduced pressure, phenol is removed and bisphenol A is concentrated. The condition of the concentration is not particularly limited. In general, the distillation is conducted at a temperature of about 100 to 170° C. and a pressure of 5 to 70 kPa. When the temperature is lower than 100° C., a high vacuum is necessary. When the temperature exceeds 170° C., removal of excessive heat is necessary in the following step of crystallization. Therefore, a temperature outside the above range is not preferable. It is preferable that the concentration of bisphenol A in the concentrated residual liquid is in the range of 20 to 50% by weight and more preferably in the range of 20 to 40% by weight. When the concentration is smaller than 20% by weight, the recovery of bisphenol A is small. When the concentration exceeds 50% by weight, transfer of a slurry obtained by the crystallization becomes difficult.

(D) Step of Crystallization and Solid-Liquid Separation

In the step of crystallization and solid-liquid separation, the adduct of bisphenol A and phenol in relative amounts of 1:1 (occasionally, referred to as the phenol adduct) is crystallized and separated from the concentrated residual liquid obtained in the step of concentration of step (C).

In the present step, the concentrated residual liquid is cooled to about 40 to 70° C. to crystallize the phenol adduct and a slurry is obtained. The cooling may be conducted by using a heat exchanger at the outside or in accordance with the crystallization with cooling under vacuum in which water is added to the concentrated residual liquid and the cooling is achieved by utilizing the latent heat of vaporization of water under a reduced pressure. In the crystallization with cooling under vacuum, water in an amount of about 3 to 20% by weight is added to the concentrated residual liquid and the crystallization is conducted, in general, under the condition of a temperature of 40 to 70° C. and a pressure of 4 to 16 kPa. When the amount of water is smaller than 3% by weight, the ability of removing the heat is insufficient. When the amount of water exceeds 20% by weight, the loss of bisphenol A by dissolution increases. Therefore, an amount of water outside the above range is not preferable. When the temperature of the crystallization is lower than 40° C., there is the possibility that the liquid for the crystallization has an increased viscosity or is solidified. When the temperature of the crystallization exceeds 70° C., the loss of bisphenol A by dissolution increases. Therefore, a temperature outside the above range is not preferable.

In the present invention, the slurry containing the phenol adduct obtained by the crystallization is separated into the phenol adduct and the mother liquor of the crystallization containing byproducts by filtration. Since the fraction of substitution with the solvent can be increased in the filtration in comparison with the centrifugation during the washing, impurities on the surface of crystals and between crystals can be effectively removed. A portion of the mother liquor of crystallization containing fine crystals passing through the filter may be recycled to the reactor without further treatments. At least a portion of the mother liquor may be treated with an alkali so that phenol and isopropenylphenol are recovered. A portion or the entire amount of the mother liquor may be treated for isomerization and recycled to the raw materials of crystallization.

By filtering phenol slurry of bisphenol A (1) which contains the adduct of bisphenol A and phenol in the crystalline condition with a filter, a layer of the adduct of bisphenol A and phenol in the crystalline condition is formed on the filter. The layer of the adduct is then washed with a washing liquid. As the washing liquid, phenol recovered in the step of concentration of step (C) or a washing liquid obtained after being used in the dissolution, crystallization and solid-liquid separation of the adduct of bisphenol A and phenol of step (E) may be used.

It is preferable that the filtration and the washing are conducted under a reduced pressure of about 40 to 90 kPa so that the fraction of substitution with the solvent is increased.

It is sufficient that the filter used for the filtration has an opening which can separate the crystals. An excessively great opening and an excessively small opening both cause a decrease in the efficiently of the filtration.

To obtain a product having a high purity, it is effective that the crystallization and the solid-liquid separation are repeated in a plurality of times. In the present invention, after the step of crystallization and solid-liquid separation of step (D) and the dissolution, crystallization and solid-liquid separation of the adduct of bisphenol A and phenol of step (E) are conducted at least twice, the step of melting by heating of step (F) and the step of removal of phenol from bisphenol A of step (G) are conducted.

(E) Step of Dissolution, Crystallization and Solid-Liquid Separation of the Adduct of Bisphenol A and Phenol

The phenol adduct crystallized and separated in step (D) is dissolved into a solution containing phenol. The solution containing phenol used in the present step is not particularly limited. For example, phenol recovered in the step of concentration of step (C), the washing liquid of the phenol adduct formed in the crystallization and solid-liquid separation of step (D), the mother liquor obtained by the solid-liquid separation of the crystallized phenol adduct and the washing liquid of the phenol adduct in the present step of step (E) and steps following step (E), may be used.

In the present step, the solution containing phenol is added to the phenol adduct obtained in step (D). The phenol adduct is dissolved by heating at about 80 to 110° C. and a solution containing bisphenol A having a concentration of bisphenol A preferable for the crystallization is prepared. By dissolution and crystallization of the layer of the adduct obtained by the washing as described above, phenol slurry of bisphenol A (2) containing the adduct of bisphenol A and phenol in the crystalline condition is obtained.

Since phenol slurry of bisphenol A (2) prepared as described above has a small viscosity at a relatively low temperature, phenol slurry of bisphenol A (2) can be handled relatively easily and is suitable for filtration with a filter or for centrifugation. In the present invention, the content of liquid can be decreased in accordance with the centrifugation and the load in steps (F) and (G) and, in particular, in step (G) can be decreased.

The centrifugal force used in the centrifugation is in the range of 200 to 1,200 G and preferably in the range of 300 to 1,000 G. When the centrifugal force exceeds 1,200 G, fragmentation of the crystals takes place markedly and the loss of the crystals accompanied with the mother liquor increases markedly due to the fragmentation. When the centrifugal force is smaller than 200 G, the content of the liquid in the crystals increases markedly. Therefore, a centrifugal force outside the above range is not preferable.

As the washing liquid for the phenol adduct separated by the centrifugation, phenol recovered in the step of concentration of step (C) may be used.

The above procedures of crystallization and solid-liquid separation of the phenol adduct from the solution containing bisphenol A, dissolution of the phenol adduct using the solution containing phenol and crystallization and solid-liquid separation of the phenol adduct are conducted at least twice.

(F) Step of Melting by Heating

In the step of melting by heating, the phenol adduct crystallized and separated in step (E) described above is melted by heating. In this step, the phenol adduct is melted by heating at about 100 to 160° C. and a liquid mixture is obtained.

(G) Step of Removing Phenol

In the step of removing phenol, phenol is removed by distillation under a reduced pressure and bisphenol A in the melted condition is recovered. The above distillation under a reduced pressure is conducted, in general, under the condition of a pressure in the range of 1.3 to 13.3 kPa and a temperature in the range of 150 to 190° C. The residual phenol can be further removed by steam stripping.

(H) Step of Granulation

In the step of granulation, bisphenol A in the melted condition obtained in step (G) described above is formed into liquid droplets, cooled, solidified and formed into a product by a granulating apparatus such as a spray drier. The liquid droplets are formed by spraying or scattering and are cooled with nitrogen or the air.

The process for producing bisphenol A of the present invention is characterized in that the filtration is used in the step of crystallization and solid-liquid separation of step (D) and the layer of the adduct of bisphenol A and phenol in the crystalline condition formed on the filter is washed with the washing liquid. Due to this operation, the fraction of substitution with the solvent can be increased and impurities on the surface of the crystals and between the crystals are effectively removed. Since the centrifugation is used in the step of dissolution, crystallization and solid-liquid separation of step (E), the content of the liquid is decreased and the load in the step of melting by heating of step (F) and in the step of removing phenol from bisphenol A of step (G) can be decreased.

As described above, when bisphenol A is separated from the reaction mixture, the adduct of bisphenol A and phenol can be efficiently recovered from the mother liquor at a high purity in accordance with the present invention.

The present invention will be described more specifically with reference to examples in the following. However, the present invention is not limited to the examples.

### PREPARATION EXAMPLE

To a reactor packed with 600 g of a cation exchange resin, phenol at a rate of 4,600 g/hr, acetone at a rate 280 g/hr and ethylmercaptan at a rate of 16 g/hr were supplied continuously while the temperature was kept at 75° C. The reaction mixture was transferred to the step of removing substances having low boiling points so that substances having low boiling points containing unreacted acetone as the main component were removed. A reaction product after the step of removing substances having low boiling points which contained bisphenol A and phenol as the main components was obtained at a rate of 4,640 g/hr. From the reaction product, a portion of phenol was removed under the condition of a temperature of 165° C. and a pressure of 53.3 kPa and the resultant solution was concentrated so that the concentration of bisphenol A was adjusted at 30% by weight. To the concentrated solution of bisphenol A, water was added and the crystallization is conducted by cooling at the temperature of 45° C. under stirring. Thus, an adduct of bisphenol A and phenol was separated by crystallization.

### Example 1

The slurry of bisphenol A and phenol obtained in Preparation Example 1 in an amount of 3,000 g was poured into a filter having a stainless steel net having an opening of 63 μm and heated at about 60° C. and filtered under suction for 60 seconds. The obtained wet cake had a thickness of about 83 mm. A portion of the wet cake was taken, washed with hexane in the same amount by weight and dried under a reduced pressure at the room temperature for 24 hours and the weight of the dried adduct was measured. As the result, the content of the liquid was found to be 24.6%. Separately, 300 g of the wet cake was taken and placed on a filter having a stainless steel net having an opening of 63 μm and heated at about 45° C. and 75 g of melted phenol at about 50° C. was poured into the filter approximately uniformly. After phenol was kept for 10 seconds, filtration was conducted under sucking at a pressure of 80 kPa. This operation was repeated once. The amounts of the 2,4-isomer in the filtrate and in a liquid obtained by dissolving the wet cake were determined in accordance with the liquid chromatography and it was found that 98.4% or more had been substituted. The wet cake was dissolved into 250 g of phenol and 6 g of water at 95° C. The obtained solution had a Harzen index of APHA5 at 175° C. and 20 minutes. The crystallization was conducted by cooling the solution at 45° C. under stirring and a slurry containing the adduct of bisphenol A and phenol was obtained.

To a cylinder made of stainless steel, a metal net having an opening for filtration of 100 μm was attached. Into the cylinder heated at 50° C. in advance, the slurry obtained above was placed and the cylinder was rotated at the maximum centrifugal force of 400 G for 40 seconds. After the prescribed time, the rotation of the cylinder was stopped and the content of the liquid was measured and found to be 4.2% by weight. The obtained cake was impregnated with melted phenol (50° C.) in an amount of about one half of the amount of the slurry and the cylinder was rotated at the maximum centrifugal force of 400 G for 20 seconds. The amount of the 2,4-isomer in the filtrate was measured and it was found that about 100% of the 2,4-isomer in the adduct of bisphenol A and phenol had been removed. After phenol was removed by distillation under a reduced pressure of 10 kPa (at a temperature of 167° C.), the amount of the isomer in bisphenol A was measured and found to be 0.025%.

### Comparative Example 1

The slurry obtained in Preparation Example 1 was placed into the centrifugal cylinder described in Example 1 and the cylinder was rotated at the maximum centrifugal force of 400 G for 20 seconds. The obtained wet cake had a content of the liquid of 5.7% and the fraction of substitution was 82%. The wet cake was dissolved into 250 g of phenol and 6 g of water at 95° C. After cooling and crystallization at 45° C. under stirring, the resultant product was subjected to the centrifugation at the maximum centrifugal force of 400 G for 20 seconds. The content of the liquid in the obtained wet cake was measured and found to be 4.3%.

After phenol was removed by distillation under a reduced pressure in accordance with the same procedure as that in Example 1, the amount of the 2,4-isomer in bisphenol A was measured and found to be 0.2%.

As described above, when the solid-liquid separations in step (D) and step (E) were conducted both in accordance with the centrifugation, the adduct of bisphenol A and phenol could not be recovered from the mother liquor of the reaction at a high purity.

### Comparative Example 2

The slurry obtained in Preparation Example was filtered in accordance with the same procedures as those described in Example 1. The obtained wet cake was dissolved into 255 g of phenol and 6 g of water at 95° C. After cooling and crystallization at 45° C. under stirring, the resultant product was filtered through a metal net having the same opening and washed once. The obtained wet cake had a content of the liquid of 23.8%. After phenol was removed by distillation under a reduced pressure in accordance with the same procedure as that in Example 1, the amount of the 2,4-isomer in bisphenol A was 0.020%.

As described above, when the solid-liquid separations in step (D) and step (E) were conducted both in accordance with the filtration, the obtained adduct of bisphenol A and phenol had a great content of the liquid and the load in the step of melting by heating of step (F) and, in particular, in the step of removal of phenol in bisphenol A of step (G) increased.

### Example 2

In place of the stainless steel net used in the filtration in Example 1, a filter cloth made of polypropylene having a thickness of 1.3 mm and a degree of air permeation of 73 ml/cm² (manufactured by DAIWA BOSEKI Co., Ltd.) was used in the filtration of the first step. The obtained wet cake had a content of the liquid of 25.4% and a fraction of substitution of the 2,4-isomer of 99%. The adduct of bisphenol A and phenol obtained after the centrifugation had a content of the liquid of 4.0%. After phenol was removed by distillation under a reduced pressure in accordance with the same procedures as that in Example 1, the amount of the 2,4-isomer in bisphenol A was 0.03%.

### INDUSTRIAL APPLICABILITY

In accordance with the process for producing bisphenol A of the present invention, the fraction of substitution with the solvent in the step of crystallization and solid-liquid separation of step (D) is increased and impurities on the surface of the crystals and between the crystals can be effectively removed. The content of the liquid in the step of dissolution, crystallization and solid-liquid separation of the phenol adduct of step (E) is decreased and the load in the step of melting by heating of step (F) and in the step of removing phenol in bisphenol A of step (G) can be decreased.

Therefore, in accordance with the present invention, the adduct of bisphenol A and phenol can be recovered from the mother liquor of the reaction efficiently at a high purity.

1. A process for producing bisphenol A which comprises crystallizing an adduct of bisphenol A and phenol from a phenol solution of bisphenol A which is obtained by reacting phenol and acetone in a presence of an acid catalyst, treating a formed slurry by solid-liquid separation and removing phenol from a solid component, wherein the process comprises forming a layer of the adduct of bisphenol A and phenol in a crystalline condition on a filter by filtering phenol slurry of bisphenol A (1) comprising the adduct of bisphenol A and phenol in a crystalline condition with the filter, washing the layer of the adduct with a washing liquid, dissolving the layer of the adduct obtained after the washing in a solution comprising phenol, producing phenol slurry of bisphenol A (2) comprising the adduct of bisphenol A and phenol in a crystalline condition by crystallization, and separating the adduct of bisphenol A and phenol in a crystalline condition by centrifugation of phenol slurry of bisphenol (2).

2. A process for producing bisphenol A according to claim 1, wherein at least one of the filtration of phenol slurry of bisphenol A (1) and/or the washing of the layer of the adduct of bisphenol A and phenol is conducted under a reduced pressure.

3. A process for producing bisphenol A according to claim 2, wherein at least one of the filtration of phenol slurry of bisphenol A (1) and/or the washing of the layer of the adduct of bisphenol A and phenol is conducted under a pressure of 40 to 90 kPa.

4. A process for producing bisphenol A according to claim 1, wherein phenol recovered in a step of concentrating bisphenol A is used as the washing liquid.

5. A process for producing bisphenol A according to claim 1, wherein washing water obtained after being used in a step of dissolution, crystallization and solid-liquid separation of the phenol adduct is used as the washing liquid.

6. A process for producing bisphenol A according to claim 1, wherein a portion of a mother liquor of crystallization is recycled to a reactor for the reaction of phenol and acetone.

7. A process for producing bisphenol A according to claim 1, wherein at least a portion of a mother liquor of crystallization is treated by decomposition with an alkali and recovered as phenol and isopropenylphenol.

8. A process for producing bisphenol A according to claim 1, wherein at least a portion of a mother liquor of crystallization is isomerized and recycled to a raw material of crystallization.

9. A process for producing bisphenol A according to claim 1, wherein the filter is a filter having a stainless steel net or a filter cloth made of polypropylene.

10. A process for producing bisphenol A according to claim 1, wherein the centrifugal force used in the centrifugation is in the range of 200 to 1200G.
